(19) United States
(12) Patent Application Publication
(10) Pub. No.: US 2021/0176996 A1
(43) Pub. Date: Jun. 17, 2021

(54) ENHANCED NON-HORTICULTURE FOOD SUPPLY BIO-SECURITY SYSTEM

(71) Applicant: Beneficial Biome Solutions, Inc, St. Louis, MO (US)

(72) Inventors: Douglas Steven Pernikoff, Glencoe, MO (US); Grant Lee Weber, St. Louis, MO (US)

(73) Assignee: Beneficial Biome Solutions, Inc, St. Louis, MO (US)

(21) Appl. No.: 17/123,688

(22) Filed: Dec. 16, 2020

Related U.S. Application Data
(60) Provisional application No. 62/949,001, filed on Dec. 17, 2019.

(51) Int. Cl. A01N 65/40; A61K 36/889; A01N 65/22; A61K 36/537; A01N 63/10; A61K 35/612; A61K 36/53; A61K 36/23; A01N 65/10; A01N 65/16; A61K 36/45; A01N 65/48; A61K 36/9068; A01N 65/20; A61K 36/48; A61K 36/07; A01N 65/00; A01N 65/24; A61K 36/54; A01N 65/36; A61K 36/752; A01N 65/03; A61K 36/02; A23L 3/3472; A23L 3/3571; A23B 4/22

(52) U.S. Cl. CPC A01N 65/40; A23V 2002/00; A01N 65/22; A61K 36/537; A01N 63/10; A61K 35/612; A61K 36/53; A61K 36/23; A01N 65/10; A01N 65/16; A61K 36/45; A01N 65/48; A61K 36/9068; A01N 65/20; A61K 36/48; A61K 36/07; A01N 65/00; A01N 65/24; A61K 36/54; A01N 65/36; A61K 36/752; A01N 65/03; A61K 36/02; A23L 3/3472; A23L 3/3571; A23B 4/22; A61K 36/889

(57) ABSTRACT

We have created a novel formulation to serve as a sanitizing, disinfectant or cleanser, excluding traditional synthetic chemicals historically addressing this role. The product is all-natural, GRAS certified ingredients that is safe, non-toxic and can be inhaled, ingested, applied topically to the animal body and of course, to all other surfaces we have defined herein. Thereby safe for water bowls, water and feed troughs, and water line flushing and disinfecting/sanitizing or cleaning.

### CROSS-REFERENCE TO RELATED APPLICATIONS

This application is a Non-Provisional application claiming priority of the Provisional U.S. Patent Application No. 62/949,001 filed on Dec. 17, 2019. This application is a continuation-in-part of the Provisional U.S. Patent Application. No. 62/891,261, which was filed on Aug. 23, 2019. This application is a continuation-in-part of Non-Provisional U.S. patent application Ser. No. 17/001,440 entitled “Anti-microbial Amalgamate of Non-toxic Native Composition,” which was filed Aug. 24, 2020. These applications are included herein in entirety by reference,

### STATEMENT REGARDING FEDERALLY SPONSORED RESEARCH OR DEVELOPMENT

Not Applicable.

The present disclosure relates to the biosecurity of non-horticulture food supply, fixtures, equipment, facilities, bio-security system, and compositions security systems. The composition is non-toxic to vertebrate animals, including humans.

### BACKGROUND

Several foods become infected during the food supply chain system, causing various diseases in humans. Animal food and non-food products can contribute to these infectious diseases. Foods are exposed to several harsh chemicals to clean and sanitize these foods to remove infectious microbes. Although these chemicals remove dirt and mud and inactivate the disease-causing infectious microorganisms, the chemical themselves are hazardous to animal and human health. Both human food consumers and personnel or workers in the food supply chain are exposed and harmed by these chemicals via the ingestion of food, inhalation, skin absorption of the chemicals, etc. These chemicals can cause diseases that range from discomfort to life-threatening, such as skin and food allergies, respiratory infections and diseases, asthma, digestive diseases, damaged gut lining, inflammation, cancers, and others.

These chemicals can enter water systems, further adding to their hazardous toxic effects on humans, equipment, infrastructure, and the general environment. Due to their corrosive nature, these chemicals cause damage to the equipment and infrastructure. Additionally, the chemicals can react with water, dissolved salts, dissolved minerals, air, etc., to yield tremendous harm to equipment, infrastructure, the environment, human populations, and others. The invention herein alleviates these chemicals' toxin effects yet provides complete protection for humans and animals from infectious microbes.

### BRIEF DESCRIPTION OF THE INVENTION

This application relates to a novel and unique system and composition used during the non-horticulture food-supply, fixtures, equipment, facilities bio-security system, and compositions to maintain bio-security without hazardous and toxic effects in humans, equipment, infrastructure, ecosystem, and environment.

The novel food chain bio-security system and composition may serve as a disinfectant or NCDAI, excluding traditional synthetic chemicals historically addressing this role. The native composition comprises GRAS-certified ingredients that may be safe, non-toxic, inhaled, ingested, applied topically to the human and animal body, and inanimate surfaces. The system and composition may be safe for water bowls, water, feed troughs, and water in line flushing and disinfecting systems.

The system and composition of the invention disclosed herein may be used in non-horticulture food supply systems.

The bio-security system and composition disclosed herein may apply to the inhibition of disease transference between food animals, non-food animal products, zoonoses, humans handling animals or animal products, and human end consumers.

The bio-security system and composition disclosed herein may apply to the inhibition of disease transference between humans and various animals such as farm animals, domesticated animals, sports animals, zoo animals, pets, laboratory animals, wild animals.

The bio-security system and compositions may contain at least two or more native and/or non-chemical substances. The native and/or non-chemical substances may be selected from black cumin oil, carob powder, cardamom powder, fenugreek powder, moringa, Agaricus mushroom powder, micro-algae, and insect protein powder. These can be used in powder, oil, extract form, or any combination thereof.

The bio-security system and compositions may be at least one or more native substances selected from coriander powder, ginger powder, cinnamon powder, cranberry seed oil, and oregano oil.

In an embodiment of this invention, the composition may kill microbes or retard the microorganisms' growth (disinfects) immediately on contact.

In an embodiment of this invention, the composition may disinfect for longer times, such 0 to 14 days, 0 to 28 days, 0-30 days, 0-1.5 months, 0-2 months, 0-2.5, 0-3, 0-3.5, 0-4, 0-4.5, 0-5, 0-5.5, 0-6 months, 0-7, 0-8, 0-9, 0-10, 0-11, 0-12 months, or a combination of thereof.

In an embodiment of the composition of this invention, the composition may be stable on inanimate and animate surfaces for short, e.g., 0.5, 1, 2, 3, 4, 5, 6, 7, 8, 9, 10, 11, 12, 13, 14, 15, 16, 17, 18, 19, 20, 21, 22, 23, 24 hour or long e.g., 0-1, 0-2, 0 to 14 days, 0 to 28 days, 0-30 days, 0-1.5 months, 0-2 months, 0-2.5, 0-3, 0-3.5, 0-4, 0-4.5, 0-5, 0-5.5, 0-6 months, 0-7, 0-8, 0-9, 0-10, 0-11, 0-12, 0-13, 0-14, 0-15, 0-16, 0-17, 0-18, 0-19, 0-20, 0-21, 0-22, 0-23, 0-24, 0-25, 0-26, 0-27, 0-28, 0-29, 0-30 months, and a combination of thereof.

In an embodiment of this invention, the system and composition may be non-toxic, non-corrosive, non-irritant, inhalable, and/or ingestible.

In an embodiment of this invention, the system and composition may have a surfactant, emulsifier, or both properties.

In an embodiment of this invention, the system and composition may not contain any surfactant, emulsifier, or both.

In an embodiment of this invention, the system and composition may have froth.

In an embodiment of this invention, the system and composition may have less than 40%, 50%, 60%, 70%, 80%, 90%, and/or 100% froth than similar synthetic compositions on the market.

In an embodiment of this invention, the system and composition may not contain any froth, i.e., 0%, 1%, 2%, 3%, 4%, or 5% less froth than similar synthetic composition the market.

In an embodiment of this invention, the system and composition have a positive environmental impact.

In an embodiment of this invention, the system and composition may have a neutral environmental impact, i.e., neither positive nor negative environmental impact.

In an embodiment of this invention, the composition may contain a flavoring, fragrance, and/or color agent.

In an embodiment of this invention, the composition is non-foaming, e.g., zero foam.

In an embodiment of this invention, the systems and composition may have anti-coccidial, anti-mite, anti-arthropod, anti-insect, e.g., fleas, lice, beetles, anti-bacterial, anti-yeast, anti-fungus, anti-viral, parasiticides, arthrocides, pesticide, anti-microorganisms, anti-microbials, anti-rickettsial, and any combination of thereof.

The systems and composition of this invention may be applied as a spray, pressurized-spray, atomized spray, wash, rinse, hand-held spray, hose, shower, mist, aerosol, nebulizer, spray gun, jet-spray, vaporize, sprinkle, pressurized canister spray, containers with pump spray top, containers with pump device or mix, apply into commercial power spray units, and, integrated into water line pressure cleaning systems.

This invention's composition may be manufactured and/or shipped in concentrated form and diluted with coconut oil, MCT oil, or both at any site of use.

The composition of this invention may be a mixture in which solid, liquid, or gas particles are dispersed throughout the bulk of a fluid or gas.

In an embodiment of the invention, the composition may be a suspension, a mixture, a gel, a colloidal solution, a solution, emulsion, aerosol, aerogel, spray, or any combination thereof.

In an embodiment of this invention, the system and composition may be used to disinfect inanimate surfaces, animate surfaces, waters, water-lines, air, vents, bins hatcheries, nest, physical support structures, incubators, premise, caged layer housing, broiler house, backyard poultry coups, HVAC, floors, ancillary structural fixtures, personnel clothing, glasses, and other protective equipment, personnel exposed skin, hair, nasal passages, and others.

In an embodiment of this invention, the system and composition may be used in any vertebrate housing as a topical on livestock, like an egg cleanser, onto husbandry fixtures (cages, nest boxes, incubators, coops, flooring, walls, premise-penned outside yards, large commercial holding for broilers or layers), tool surfaces and any combination thereof.

In an embodiment of this invention, the composition may be packaged in polypropylene 8 oz hand sprayers, 1.6 kg jugs with a commercial hand pump sprayer or 55-gallon drums support a universal adapter for any/all range of large volume commercial sprayer units.

Non-horticulture food supply bio-security systems method, wherein disinfecting and preventing the passage of microbial from a food-vertebrate and/or vertebrate products to human food supply chain workers.

A method of non-horticulture food supply bio-security systems, wherein disinfecting and preventing the passage of microbial from a food-vertebrate and food-vertebrate product to first human and end human consumer. The first human is the first human who handles the food-animal or food-animal product, and the end-use the human consumer.

A method of non-horticulture food supply bio-security systems, wherein disinfecting and preventing the passage of microbial zoonoses from first food vertebrate and food-vertebrate product to second food vertebrate and food vertebrate product to end human consumer

The vertebrate animal and vertebrate animal products are poultry, cattle, swine, ovine, or equine animals, farm animals, domesticated animals, sports animals, zoo animals, pets, laboratory animals, wild animals, or any combination thereof.

### BRIEF DESCRIPTION OF DRAWINGS

FIG. 1: Composition A/B/C (1:100× dilution) inhibition of Salmonella and E. coli:

FIG. 2: Composition A/B/C (1:100× Dilution) Inhibition of Campylobacter jejuni

FIG. 3. Composition A/B/C (1:100× Dilution) Inhibition of Candida albicans.

FIG. 4. Composition A/B/C Inhibition of Aspergillus fumigatus.

FIG. 5, Composition A/B/C Inhibition of (Anti-Microbial Resistance) AMR. Salmonella.

FIG. 6: Generalize Steps in Animal Slaughtering.

### DETAILED DESCRIPTION OF THE INVENTION

Examples embodiments are provided so that this disclosure will be thorough and will fully convey the scope to those who are skilled in the art. Numerous specific details are set forth, such as examples of specific components, devices, and methods, to provide a thorough understanding of embodiments of the present disclosure. It will be apparent to those skilled in the art that specific details need not be employed, that example embodiments may be embodied in many different forms and that neither should be construed to limit the scope of the disclosure. In some example embodiments, well-known processes, well-known device structures, and well-known technologies are not described in detail.

The terminology used herein is for the purpose of describing particular example embodiments only and is not intended to be limiting. As used herein, the singular forms “a,” “an,” and “the” may be intended to include the plural forms as well, unless the context clearly indicates otherwise. The terms “comprises,” “comprising,” “including,” and “having,” are inclusive and therefore specify the presence of stated features, integers, steps, operations, elements, and/or components but do not preclude the presence or addition of one or more other features, integers, steps, operations, elements, components, and/or groups thereof. The method steps, processes, and operations described herein are not to be construed as necessarily requiring their performance in the particular order discussed or illustrated unless specifically identified as an order of performance. It is also to be understood that additional or alternative steps may be employed.

Although the terms first, second, third, etc., may be used herein to describe various elements, components, regions, layers and/or sections, these elements, components, regions, layers and/or sections should not be limited by these terms. These terms may be only used to distinguish one element, component, region, layer, or section from another region, layer, or section. Terms such as “first,” “second,” and other numerical terms when used herein do not imply a sequence or order unless clearly indicated by the context. Thus, a first element, component, region, layer, or section discussed below could be termed a second element, component, region, layer, or section without departing from the teachings of the example embodiments.

Spatially relative terms, such as “inner,” “outer,” “beneath,” “below,” “lower,” “above,” “upper,” and the like, may be used herein for ease of description to describe one element or feature's relationship to another element(s) or feature(s) as illustrated in the figures. Spatially relative terms may be intended to encompass different orientations of the device in use or operation in addition to the orientation depicted in the figures. For example, if the device in the figures is turned over, elements described as “below” or “beneath” other elements or features would then be oriented “above” the other elements or features. Thus, the example term “below” can encompass both an orientation of above and below. The device may be otherwise oriented (rotated 90 degrees or at other orientations) and the spatially relative descriptors used herein interpreted accordingly.

The preceding summary, as well as the following detailed description of certain embodiments, will be better understood when read in conjunction with the appended figure of experimental data and results. As used herein, an element or step recited in the singular and proceeded with the word “a” or “an” should be understood as not excluding the plural of said elements or steps unless such exclusion is explicitly stated. Furthermore, references to “one embodiment” or “an embodiment” are not intended to be interpreted as excluding the existence of additional embodiments that also incorporate the recited features. Moreover, unless explicitly stated to the contrary, embodiments “comprising” or “having” an element or a plurality of elements having a particular property may include additional such elements not having that property. When a definition is provided herein, it supersedes any other meaning or definition.

As used herein, all percentages are expressed in weight by volume, unless otherwise stated.

Disinfectant, according to CDC, is a chemical agent used on inanimate objects (i.e., nonliving) (e.g., floors, walls, sinks) to destroy pathogenic microorganisms. However, as used herein, the term “disinfectant” means a non-chemical composition with an anti-pathogenic microorganism activity, both microbial-cidal and microbial-static activities. It may be used on both living or animate and non-living or inanimate objects, wherein the objects are liquid, gaseous, solid, and any combination thereof. Thus, Non-Chemical Disinfectant with activity on Animate and Inanimate objects (NCDAI).

As used herein, the term “microorganisms” or “microbial” means a microscopic organism that is either single/multi-cellular cellular, such as, but not limited to, virus, bacteria, fungi, yeasts, or multi-cellular such as, but not limited, ectoparasites, nematodes, ticks, lice, worms, etc.

As used herein, the term “anti-microbial” means a composition that has properties of microbial-cidal, anti-microbial-static, or a combination of both.

As used herein, the term “froth” or “foam” means the formation of bubbles by the agitation or spraying of a liquid similar to a detergent or detergent-containing liquids.

As used herein, the term “substantially froth-free or non-foaming” means that 10% or fewer bubbles are formed as compared to similar commercially available detergents and/or chemicals containing compositions.

As used herein, the term “pathogen” or “parasite” means a first organism that harbors within or on another second organism such that the pathogen or the parasite benefits at the expense of the second organism or the host, wherein the second organism is harmed.

As used herein, the term “vertebrate” means an organism that has a back-bone or vertebrate.

As used herein, the term “native” means non-synthetic and non-chemical substances found in the ecosystem.

As used herein, the term “GRAS” means generally recognized as safe is a United States Food and Drug Administration.

As used herein, the term “food supply” or “food supply chain” means a system or process of housing, feed, slaughtering, processing, packaging, shipping, storing, and selling the food or non-food products to end human users.

As used herein, the term “bio-security” means maintaining the safety and security of biological products (food and non-food) from pathogenic contamination.

As used herein, the term “personnel” means workers handling the vertebrate animals, animal products, by-products, or any combination thereof during the food supply chain.

As used herein, the term “inanimate” means nonliving materials, such as water, wood, plastic, cement, leather, hide, and others

As used herein, the term “animate” means living entities, such as animals (including humans), animal tissues, animal organs, and others.

As used herein, the term “organic” means without pesticides, chemical fertilizers, synthetic antibiotics, and non-GMO and certified organic by the U. S. Department of Agriculture (USDA).

This application relates to a novel and unique system and composition used during the food supply chain system to maintain bio-safety without hazardous and toxic effects in humans, equipment, infrastructure, ecosystem, and the environment. The microorganism contamination may occur by a variety of pathways, such as but not limited to, unclean and non-sterile environment, exposure of internal organ microbes to the environment via feces or vomiting, slaughtering, etc., personnel or workers exposure during slaughtering, animal to animal contact, animal to personnel/worker contact, personnel to personnel contact, personnel to non-work human contact, and others.

The novel food chain bio-security system and composition serve as a disinfectant or NCDAI, excluding traditional synthetic chemicals historically addressing this role. The native composition comprises GRAS certified ingredients that may be safe, non-toxic, inhaled, ingested, applied topically to the human and animal body, inanimate surfaces, etc. The system and composition may be safe for water bowls, water, feed troughs, water line flushing and disinfecting systems, etc.

This invention may prevent or retard microbial transmission from any non-human vertebrate to humans, humans to any non-human vertebrates, between humans, and the microbes may transmit the pathogen between different two or more species or the same species.

Essential oils normally may have no independent pesticidal activity when included in anti-microbial products; these products contain one or more other chemicals that perform as the active ingredients. Essential oils in anti-microbial products must instead be classified as inert ingredients and/or must be deleted from the product formulations.

In an embodiment of the invention, the composition may be applied over living or inanimated solid surfaces, in liquids, and gases, such as but not limited to, animals, animal parts, animal tissues, animal organs, equipment, water in water lines and systems, animal housing, animal housing air, slaughtering facilities, personnel, and others.

The system and composition of the invention disclosed herein may be used in non-horticulture food supply systems. These facilities include, but are not limited to, farm animals, domesticated animals, sports animals, zoo animals, pets, laboratory animals, wild animals, and human personnel or workers, etc., in these facilities.

The bio-security system and composition disclosed herein may apply to disease transference prevention between food-animals (means edible animals or edible animal parts/products), non-food animal products (such as but not limited to leather, tusk, etc.), zoonoses, or humans handling animals or animal products and human end consumers.

The bio-security system and composition disclosed herein may apply to disease transference prevention between humans and various animals such as farm animals, domesticated animals, sports animals, zoo animals, pets, laboratory animals, wild animals, etc.

The bio-security system and compositions may include at least two or more native substances. The native substances may be selected from black cumin oil, carob powder, cardamom powder, fenugreek powder, moringa, Agaricus mushroom powder, micro-algae, and insect protein powder. These may be used in powder, oil, extract form, aerosol, etc., or any combination thereof.

The bio-security system and compositions may contain at least one or more native substances selected from coriander powder, ginger powder, cinnamon powder, cranberry seed oil, and oregano oil. These may be used in powder, oil, extract form, aerosol, or any combination thereof.

In an embodiment of this invention, the composition disinfects immediately on contact. The composition renders parasitic microorganisms ineffective or inactive on contact such that the composition becomes function on contact, such as but not limited to, microseconds, milliseconds, seconds, and/or 1 to 10, e.g., 1, 2, 3, 4, 5, 6, 7, 8, 9, 10, minutes, or for longer times 15, 20, 25, 30, 35, 40, 45, 50, 55, 60 minutes, 1-24 hours and/or any combination thereof.

In an embodiment of this invention, the composition may be stable (i.e., not inactivated or destroyed) disinfects for long periods, such as up to (that is from zero) and including 1 day, 2 days, 3 days, 4 days, 5 days, 10 days, 14 days, 15 days, 20 days, 25 days, 28 days, 30 days, 35 days, 40 days, 45 days, 50 days, 55 days, 65 days, 1 month, 1.5 months, 2 months, 2.5 months, 3 months, 3.5 months, 4 months, 4.5 months, 5 months, 5.5 months, 6 months, 6.5 months, 7 months, 7.5 months, 8 months, 8.5 months, 9 months, 9.5 months, 10 months, 10.5 months, 11 months, 11.5 months, 1 year, 1.5 years, 2 years and/or any combination of thereof.

In an embodiment of the composition of this invention, the composition may be stable on both inanimate and animate surfaces for short and/or long periods. The composition may be applied one time and may remain on the inanimate and animate surfaces for short and/or long periods. These periods include, but not limited to, up to (i.e., zero) microseconds, milliseconds, seconds, and/or up to 60 minutes, 1-24 hours, 1 day, 2 days, 3 days, 4 days, 5 days, 6.7, 8, 9, 10 days, 14 days, 15 days, 20 days, 25 days, 28 days, 30 days, 35 days, 40 days, 45 days, 50 days, 55 days, 65 days, 1 month, 1.5 months, 2 months, 2.5 months, 3 months, 3.5 months, 4 months, 4.5 months, 5 months, 5.5 months, 6 months, 6.5 months, 7 months, 7.5 months, 8 months, 8.5 months, 9 months, 9.5 months, 10 months, 10.5 months, 11 months, 11.5 months, 1 year, 1.25 years, 1.5 years, 1.75 years, 2 years or any combination of thereof.

In an embodiment of the composition of this invention, the system and composition may be non-toxic, non-corrosive, non-irritant, inhalable, and ingestible for both inanimate and animate surfaces. Hence, the composition may possess no toxic and irritant effects on human personnel. In fact, direct contact with skin, eyes, ears, hair, nails, or nasal, oral, or digestive mucosa causes no discomfort, sickness, and/or disease.

In an embodiment of the composition of this invention, the system and composition may be non-toxic Thus, may prevent harmful effects, such as but are not limited to, burning sensation, confusion, drowsiness, fainting fits, loss of consciousness, coma, tissue destruction, vomiting, indigestion, stomach pain, skin-bruising, irritation of skin, eyes, and other parts of the body, blurry vision, coughing, nausea, shortness of breath, watery eyes, pain in the chest, throat, nose, eye irritation, wheezing, pneumonia, fluid in the lungs, irritation, inflammation, rash, etc. Thus, non-toxic means without such toxic effects in any vertebrates.

In an embodiment of this invention's composition, the system and composition may have a neutral environmental impact. Because it is non-toxic, non-corrosive, non-irritant, and bio-degradable, the composition may cause no additional pollution in the environment.

In an embodiment of this invention's composition, the system and composition may have a positive environmental impact. When present in the environment, ecosystems, water bodies, soil, air, and others, the anti-microbial activity may inactivate the microorganisms, and thus, may cause a positive environmental impact.

In an embodiment of the composition of this invention, the composition may contain flavoring, fragrance, coloring agents, or any combination thereof, such as but not limited to cinnamon, mint, peppermint, spearmint, menthol, basil, clove, cilantro, fennel, eucalyptus, garlic, lavender, lemon, lemon-grass, lemon oil, citrus, orange, rose, sage, etc., and combination thereof. These plants may be in powder, oil, extract, solid, liquid, resin, vapor forms, etc., and/or any combination thereof.

In an embodiment of the composition of this invention, the composition may be non-foaming and lacks detergent-like effects of the formation of foam or froth on agitation.

In an embodiment of the composition of this invention, the systems and composition may have anti-coccidial, anti-mite, anti-arthropod, anti-insect (e.g., fleas, lice, beetles), anti-bacterial, anti-yeast, anti-fungus, anti-viral, parasiticides, arthrocides, pesticide, anti-microbials, anti-rickettsial, etc., and/or any combination of thereof.

The systems and composition of this invention may be applied as a spray, pressurized-spray, atomized spray, wash, rinse, hand-held spray, hose, shower, mist, aerosol, nebulizer, spray gun, jet-spray, vaporize, sprinkle, pressurized canister spray, containers with pump spray top, containers with pump device or mix, apply into commercial power spray units, and, integrated into water line pressure cleaning system, etc., and/or any combination thereof.

The composition of this invention may be manufactured and/or shipped in concentrated form and diluted with a dilutant, such as but not limited to, coconut oil or medium-chain triglyceride (MCT oil), or both at any site of use. The concentration may be 2×, 4×, 5×, 6×, 8×, 10×, 15×, 20×, 25×, or 50×, where ×means times or multiple. The dilutant may be any material such as liquid, solid, or aerosol that may dilute a mixture, amalgamate, and/or a composition.

An embodiment of any of the composition further contains at least one or more dilutant such as, but not limited to, coconut oil, peanut oil, soybean oil, corn oil, canola oil, cottonseed oil, olive oil, palm oil, rapeseed oil, safflower oil, sesame oil, pumpkin oil, almond oil, grape seed oil, avocado oil, flax-seed oil, ghee, emu oil, alcohols, sunflower, or any combination thereof. These dilutant oils may be mechanically extracted, chemically extracted, or a combination of both, and may be from a plant or an animal source. The dilutant may be liquid, solid, vapor, an aerosol, and/or any combination thereof.

Another embodiment of the composition optionally contains at least one or more thickeners such as, but not limited to, fumed silica, natural gum, starch, gelatin, agar, arrowroot, pectin, psyllium, sago, dextrin, cellulose, tapioca, tapioca starch, Xanthum gum, Kuzu/kudzu, Polyethylene glycol (PEG), waxes, etc., and/or any combination thereof.

Another embodiment of the composition further contains at least one or more solvents such as, but not limited to, isopropyl alcohol, ethanol, organic solvents, inorganic solvents, polar solvents, non-polar solvents, water, oils, alcohols, etc., and/or any combination thereof.

This invention's composition may be a mixture in which solid, liquid, or gas particles are dispersed throughout the bulk of a fluid and/or gas.

In an embodiment of the invention, the composition may be a suspension, a mixture, a gel, a colloidal solution, a solution, emulsion, aerosol, aerogel, spray, and/or any combination thereof.

In an embodiment of the composition of this invention, the system and composition may be used to disinfect innate surfaces, non-innate surfaces, waters, water-lines, air, vents, bins hatcheries, nest, physical support structures, incubators, premise, caged layer housing, broiler house, backyard poultry coups, HVAC, floors, ancillary structural fixtures, personnel clothing, glasses, and other protective equipment, and personnel exposed skin, hair, nasal passages, others, and/or any combinations thereof.

In an embodiment of the composition of this invention, the system and composition may be used in any vertebrate housing as a topical on livestock, like an egg cleanser, onto husbandry fixtures (cages, nest boxes, incubators, coops, flooring, walls, premise-penned outside yards, large commercial holding for broilers or layers), tool surfaces, etc., and/or any combination thereof.

In an embodiment of the system and composition, the composition may be packaged in polypropylene 8 oz hand sprayers, 1.6 kg jugs with a commercial hand pump sprayer, or in 55-gallon drums that can support a universal adapter for any/all range of large volume commercial sprayer units.

The vertebrate animal and vertebrate animal products may be poultry, cattle, swine, ovine, equine animals, farm animals, domesticated animals, sports animals, zoo animals, pets, laboratory animals, wild animals, or any combination thereof.

The composition of this invention may be used to prevent a variety of microbial or microorganism infections in all vertebrates such as, but not limited to, farm animals, domesticated animals, sports animals, zoo animals, pets, laboratory animals, wild animals, etc., or any combination thereof. The examples of farm animals may be, but not limited to, pig, chicken, cattle, etc. Domesticated animals' examples may be, but not limited to, goat, bull, buffalo, bezoar, sheep, etc. The examples of sports animals may be, but not limited to, horse, fox, dog, etc. The examples of zoo animals may be, but not limited to, zebra, rhino, alligator, snake, lizard, frog, bird, fish, etc. The examples of pet animals may be, but not limited to, dog, cat, horse, etc. The examples of laboratory animals may be, but not limited to, rats, frogs, mice, etc. The examples of wild animals may be, but not limited to, elephants, lions, cobras, etc. These animals may be of any age, including eggs, and there may be an overlap between the aforementioned animal groups. The eggs may benefit directly by spraying the composition onto them or indirectly from their mother.

In an embodiment of the invention, a composition provides an anti-microbial activity. The composition contains a mixture of native substances, for example, herbal plants, krill, micro-algae, cranberry seed, chia, black cumin, carob, oregano, coriander, ginger, fenugreek, cinnamon, trans-cinnamaldehyde, cardamom, or any combination thereof. These plant products may be in powder, flour, oil, extract, solid, liquid, resin, diffusable, aerosol, vapor form, and/or any combination thereof.

Another embodiment of the invention further may contain other substances such as, but not limited to, Agaricus mushroom, moringa, kelp, insects, insect-parts, rosemary, mustard, fenugreek, thyme, turmeric, others, or any combination thereof. These substances may be in powder, flour, oil, extract, solid, liquid, resin, diffusable, aerosol, vapor form, and/or any combination thereof.

The process of animal slaughtering may be divided into the following subroutines: Clearance for cleaning, Assembling for cleaning, Removal of solid waste, Pre-rinsing with water, Application of detergent, Rinsing, Disinfection, Post rinsing, Post-treatment, and Disassembling after cleaning.

In an embodiment of the invention, the composition may be used and/or stored at any temperature negative 10 to 130 degrees Celsius such as −10, −9, −8, −7, −6, −5, −4, −3, −2, −1, 0, 1, 2, 3, 4, 5, 6, 7, 8, 9, 10, 15, 20, 25, 30, 35, 40, 80, 85, 90, 98, 99, 100, 101, 102, 103, 104, 105, 110, 115, 120, 125, 130, etc., and any combination thereof.

In an embodiment of the invention, the composition may or may not be used or stored under ventilation conditions, humidity, moisture, lighted, dark, day-light, artificial light, UV light, temperature, aseptic, pressure, concentrated, etc., or any combination thereof.

In an embodiment of the invention, the composition may be used in an over-head spray-system or sprinkler system, ground sprinkler system, commercial sprinkler system, automatic and/or manual washing systems, mechanical washing system, hose, buckets, etc., or any combination thereof.

In an embodiment of the invention, the composition may be used under high or low (20-120 psi) pressure such as 20, 30, 35, 40, 45, 50, 55, 60, 65, 70, 75, 80, 85, 90, 95, 100, 105, 110, 115, 120 psi, etc., and any combination thereof.

In an embodiment of the invention, the composition may be applied to the animal, animal part, human worker, clothing and protective equipment, facilities, floors, walls, roof, slaughtering equipment, instruments, systems, etc., pre and/or post washing, pre- and/or post drying, for short and/or long time periods, such as milliseconds, seconds, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50, 55, 60 minutes, 1, 1.5, 2, 2.5, 3, 3.5, 4, 5, 6, 7, 8, 9, 10 hours, any combination thereof.

In an embodiment of the invention, the composition may and/or may not contain strong, medium, week, or any oxidizing, reducing, and/or brominating agents. The composition may and/or may not contain aldehydes, ammonia, explosive, flammable, oral toxic, skin corrosive or irritant, eye irritation or damaging, acute or chronic toxic substances, chemical-smell, reactive chemicals, aquatic life-toxins, etc., and any combination thereof. The composition is non-reactive, non-oxidizing, non-reducing, non-explosive, non-flammable under ambient (normal room temperature, 25 to 35 degree Celsius, pressure, etc.) and environmental conditions.

In an embodiment of the invention, the workers handing the composition may and/or may not require special clothing, personal protective wearables and/or protective equipment, etc., and any combination thereof.

In an embodiment of the invention, the composition may have no impact on taste, texture, consistency, color, flavor, smell, etc., and/or any combination thereof.

In an embodiment of the invention, the composition may enhance taste, texture, consistency, color, flavor, smell, etc., and/or any combination thereof.

In an embodiment of the invention, the composition may be used in several stations such as pre-post-pic-spray, post pick spray, post-evisceration dip, chill, post-chill dip, etc., and any combination thereof. The composition may be part of food-animal processing and production, components of the multi-hurdle approach, multi-hurdle food safety intervention program, holding pens, animal housing, animal feeding area, multi pre-harvest, hide-on application points inside and outside the slaughtering plant, etc., and any combination thereof.

This invention's composition may be sprayed on to several innate surfaces in an embodiment of this invention. For example, stone or lava blocks, concrete, limestone or asphalt stabilized bricks, steel, aluminum, zinc, lead, copper, tin, alloys, rubber, plastic, concrete, cement, glass, painted surfaces, galvanized iron, wood, etc., and any combination thereof.

Another embodiment of this invention may be a cleaning agent with anti-microbial activity, such as but not limited to, an antiseptic, pressurize or non-pressurized spray, wash, liquid, or gel clearer, etc., or any combination thereof. An embodiment may provide hygiene and hygiene management control through the entire life cycle of any animal.

Another embodiment of this invention may be a cleaning agent with anti-mites activity, such as but not limited to, an antiseptic, pressurize or non-pressurized spray, wash, liquid or gel clearer, etc., or any combination thereof. An embodiment provides hygiene and management control through the entire life cycle of any animal.

Another embodiment of the composition may contain an effective amount of oregano oil, extract, powder, or juice 15%-40% by weight/volume, 20%-40% by weight/volume, 20%-35% by weight/volume, 23%-31% by weight/volume, 24%-30.5% by weight/volume, 23%-30% by weight/volume, 25%-27% by weight/volume, 15%-30% by weight/volume, 18%-25% by weight/volume, or any combination thereof.

Another embodiment of the composition may contains an effective amount of coconut powder, oil, extract, or juice 10%-40% by weight/volume, 15%-30% by weight/volume, 20%-35% by weight/volume, 18%-30% by weight/volume, 20%-25% by weight/volume, 21%-27% by weight/volume, or 21%-24% by weight/volume, or any combination thereof.

Another embodiment of the composition may contain an effective amount of chia flour, oil, extract, or juice 5%-30% by weight/volume, 10%-25% by weight/volume, 12%-22% by weight/volume, 13%-20% by weight/volume, 15%-20% by weight/volume, 16%-19% by weight/volume, or 16%-17% by weight/volume, or any combination thereof.

Another embodiment of the composition may contain effective amount of krill powder, oil, extract, or juice 5%-30% by weight/volume, 10%-25% by weight/volume, 12%-22% by weight/volume, 13%-20% by weight/volume, 15%-20% by weight/volume, 16%-19% by weight/volume, or 16%-17% by weight/volume, or any combination thereof.

Another embodiment of the composition may contain effective amount of micro-algae powder, oil, extract, or juice 5%-30% by weight/volume, 10%-25% by weight/volume, 12%-22% by weight/volume, 13%-20% by weight/volume, 15%-20% by weight/volume, 16%-19% by weight/volume, or 16%-17% by weight/volume, or any combination thereof.

Another embodiment of the composition may contain an effective amount of fumed silica 0.5%45% by weight/volume, 1%-12% by weight/volume, 1%40% by weight/volume, 3%-10% by weight/volume, 3%-9% by weight/volume, 4%-8% by weight/volume, or 5%-7% by weight/volume, or any combination thereof.

Another embodiment of the composition may contain an effective amount of cranberry seed powder, oil, extract, or juice 0.1%-10% by weight/volume, 0.5%-10% by weight/volume, 1%-9% by weight/volume, 1.5%-8% by weight/volume, 2.5%-7% by weight/volume, 2.5%-8% by weight/volume, 3%-7% by weight/volume, 3%-5% by weight/volume, or 3%-4% by weight/volume, or any combination thereof.

Another embodiment of the composition may contain an effective amount of coriander powder, extract, juice, or oil 0.1%-10% by weight/volume, 0.5%-9% by weight/volume, 1%-8% by weight/volume, 1.9%-7% by weight/volume, 2%-6% by weight/volume, 2%-5% by weight/volume, 2.5%-6% by weight/volume, 2.5%-5% by weight/volume, 2.5%-4 by weight/volume, or 2.5%-3.5% by weight/volume, or any combination thereof.

Another embodiment of the composition may contain an effective amount of ginger powder, oil, extract, or juice 0.1%-5% by weight/volume, 0.25%-5% by weight/volume, 0.5%-4% by weight/volume, 0.6%-3% by weight/volume, 0.6%-2% by weight/volume, 0.6%-1.5% by weight/volume, 0.6%-1% by weight/volume, or 0.5%-1% by weight/volume, or any combination thereof.

Another embodiment of the composition may contain an effective amount of black cumin powder, oil, extract, or juice 0.05%-5% by weight/volume, 0.1%-5% by weight/volume, 0.15%-4% by weight/volume, 0.2%-3% by weight/volume, 0.2%-2% by weight/volume, 0.2%-1.5% by weight/volume, 0.3%-1% by weight/volume, or 0.4%-1% by weight/volume, or any combination thereof.

Another embodiment of the composition may contain an effective amount of carob powder, oil, extract, or juice 0.1%-5% by weight/volume, 0.25%-5% by weight/volume, 0.5%-4% by weight/volume, 0.5%-3% by weight/volume, 0.5%-2% by weight/volume, 0.5%-1.5% by weight/volume, 0.5%-1% by weight/volume, and/or 0.6%-0.9% by weight/volume, or any combination thereof.

Another embodiment of the composition may contain an effective amount of fenugreek powder, oil, extract, or juice 0.01%-5% by weight/volume, 0.025%-5% by weight/volume, 0.1%-4% by weight/volume, 0.1%-3% by weight/volume, 0.1%-2% by weight/volume, 0.1%4.5% by weight/volume, 0.1%-1% by weight/volume, and/or 0.2%-0.9% by weight/volume, or any combination thereof.

Another embodiment of the composition may contain an effective amount of agaricus mushroom powder, oil, extract, or juice 0.01%-5% by weight/volume, 0.025%-5% by weight/volume, 0.1%-4% by weight/volume, 0.1%-3% by weight/volume, 0.1%-2% by weight/volume, 0.1%-1.5% by weight/volume, 0.1%-1% by weight/volume, and/or 0.2%-0.9% by weight/volume, or any combination thereof.

Another embodiment of the composition may contain an effective amount of cardamom powder, oil, extract, or juice 0.01%-5% by weight/volume, 0.025%-5% by weight/volume, 0.1%-4% by weight/volume, 0.1%-3% by weight/volume, 0.1%-2% by weight/volume, 0.1%-1.5% by weight/volume, 0.1%-1% by weight/volume, and/or 0.2%-0.9% by weight/volume, or any combination thereof.

Another embodiment of the composition may contain an effective amount of oregano oil 11.0-24%, wt/wt, e.g., 11.0, 11.5, 11.6, 11.7, 11.8, 11.9, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.1, 23.2, 23.3, 23.4, 23.5, 23.6, 23.7, 23.8, 23.9, 24% wt/wt or any combination thereof. These are in % wt/wt.

Another embodiment of the composition may contain an effective amount of coconut oil 19-24.5%, wt/wt, e.g., 19, 19.5, 19.6, 10.7, 19.8, 19.8, 19.9, 20, 20.1. 20.2, 20.3, 20.4, 20.5, 21, 21.5, 22, 22.5, 23, 23.1, 23.2, 23.3, 23.4, 23.4, 23.5, 23.6, 23.7, 23.8, 23.9, 24, 24.1, 24.2, 24.3, 24.4, 24.5 or an combination thereof. These are in % wt/wt.

Another embodiment of the composition may contain an effective amount of elantria DHA 400 algae 15.-20% wt/wt, e.g., 15.1, 15.2, 15.3, 15.4, 15.5, 15.6, 15.7, 15.8, 15.9, 16, 16.1, 16.2, 16.3, 16.5, 16.6, 16.7, 16.8, 16.9, 17, 17.5, 17.6, 17.7, 17.8, 17.9, 18, 18.1, 18.2, 18.3, 18.4, 18.5, 18.6, 18.7, 18.8, 189.9, 19, 19.5, 20, or any combination thereof. These are in % wt/wt.

Another embodiment of the composition may contain an effective amount of MCT oil 21-26.5%, wt/wt, e.g., 21, 21.1, 21.2, 21.3, 21.4, 21.5, 21.6, 21.7, 21.8, 21.9, 22, 22.1, 22.2, 22.3, 22.4, 22.5, 22.6, 22.7, 22.8, 22.9, 23, 23.5, 24, 24.5, 24.6, 24.7, 24.8, 24.9, 25, 25.1, 25.2, 25.3, 25.4, 25.5, 25.6, 25.7, 25.8, 25.9, 26, 26.5, or any combination thereof. These are in % wt/wt.

Another embodiment of the composition may contain an effective amount of cranberry see oil 2.5-4.0% wt/wt, e.g., 2.5, 2.6, 2.7, 2.8, 2.9, 3, 3.1, 3.2, 3.3, 3.4, 3.5, 3.6, 3.7, 3.8, 3.9, 4, or any combination thereof. These are in % wt/wt.

Another embodiment of the composition may contain an effective amount of coriander oil 2.-4% wt/wt, e.g., 2, 2.1, 2.2, 2.3, 2.4, 2.5, 2.6, 2.7, 2.8, 2.9, 3, 3.1, 3.2, 3.4, 3.5, 3.6, 3.7, 3.8, 3.9, 4, or any combination thereof. These are in % wt/wt.

Another embodiment of the composition may contain an effective amount of ginger oil 0.4-1.5% wt/wt, e.g., 0.4, 0.5, 0.6, 0.7, 0.8, 0.9, 1, 1.1, 1.2, 1.3, 1.4, 1.5, or any combination thereof. These are in % wt/wt.

Another embodiment of the composition may contain an effective amount of black cumin oil 0. 0.2-1.5% wt/wt, e.g., 0.2, 0.3, 0.4, 0.5, 0.6, 0.7, 0.8, 0.9, 1, 1.1, 1.2, 1.3, 1.4, 1.5, or any combination thereof. These are in % wt/wt.

Another embodiment of the composition may contain an effective amount of carob oil 0.2-1.5% wt/wt, e.g., 0.2, 0.3, 0.4, 0.5, 0.6, 0.7, 0.8, 0.9, 1, 1.1, 1.2, 1.3, 1.4, 1.5, or any combination thereof. These are in % wt/wt.

Another embodiment of the composition may contain an effective amount of fenugreek oil 0.2-1.5% wt/wt, e.g., 0.2. 0.3. 0.4, 0.5, 0.6, 0.7, 0.8, 0.9, 1, 1.1, 1.2, 1.3, 1.4, 1.5, or any combination thereof. These are in % wt/wt.

Another embodiment of the composition may contain an effective amount of agaricus oil 0.2-1.5% wt/wt, e.g., 0.2. 0.3, 0.4, 0.5, 0.6, 0.7, 0.8, 0.9, 1, 1.1, 1.2, 1.3, 1.4, 1.5, or any combination thereof. These are in % wt/wt.

Another embodiment of the composition may contain an effective amount of cinnamon oil 0.4-1.5% wt/wt, e.g., 0.4, 0.5, 0.6, 0.7, 0.8, 0.9, 1, 1.1, 1.2, 1.3, 1.4, 1.5, or any combination thereof. These are in % wt/wt.

Another embodiment of the composition may contain an effective amount of lemon oil 8.4-10.5% wt/wt, e.g., 8.4, 8.5, 8.6, 8.7, 8.8, 8.9, 9, 9.1, 9.2, 9.3, 9.4, 9.5, 9.6, 9.7, 9.8, 9.9, 10, 10.1, 10.2, 10.3, 10.4, 10.5, or any combination thereof. These are in % wt/wt.

An embodiment of the composition may be anti-bacterial, e.g., retard and/or kill all Salmonella species, E. coli, Campylobacter jejuni, etc., or any combination thereof.

An embodiment of the composition may be anti-viral, e.g., retard and/or kill Newcastle, avian influenza, Ebola, ASF virus, etc., or any combination thereof.

An embodiment of the composition may be anti-yeast, e.g., retard and or kill Candida albicans, etc.

An embodiment of the composition may be anti-fungi, e.g., retard and/or kill Aspergillus fumigatus, etc.

An embodiment of the composition may be anti-parasites, e.g., retards and/or kill all species of coccidia, etc.

An embodiment of the composition may be anti-rickettsia, e.g., retard and/or kill rickettsia, etc.

An embodiment of the composition may be anti-mites, e.g., retard and/or kill mites, etc.

An embodiment of the composition may be anti-micro-algae, e.g., retard and/or kill micro-algae, etc.

An embodiment of the composition may be anti-Salmonella species, such as S. typhimurium, S. heidelberg, S. hadar, S. seftenberg, e.g., retard and/or kill these species.

An embodiment of the composition may be anti-Clostridium perfringens; e.g., retard and/or kill Clostridium perfringens, etc

An embodiment of the composition may be anti-MRSA (methicillin resistant Staphylococcus aureus), e.g., retard and/or kill methicillin resistant Staphylococcus aureus, etc

A method of the invention may be composition spraying after simple water pressure cleaning.

A method of non-horticulture food supply bio-security systems, wherein disinfecting and preventing the passage of microbial from a food-vertebrate, vertebrate products to human worker in the food supply chain comprising:
(i) a disinfectant or NCDAI containing:
at least two or more native substances; wherein the native substances may be selected from black cumin oil, carob powder, cardamom powder, fenugreek powder, moringa, Agaricus mushroom powder, micro-algae, insect protein powder, coriander powder, ginger powder, cinnamon powder, cranberry seed oil, and oregano oil. These may be used in powder, oil, extract form, or any combination thereof
(ii) applying the said composition onto innate surfaces, non-innate surfaces, waters, water-lines, air, personnel, personnel clothing, personnel equipment, personnel protective wear, vents, personnel skin, personnel hair, personnel nails, personnel nasal passages, personnel oral passage, hatcheries, nest, physical support structures, incubators, premise, caged layer housing, broiler house, backyard poultry coups, HVAC, floors, bins, water line, pressure and non-pressure cleaning system ancillary structural fixtures and tools;
(iii) applying the said composition by one or more of the devices selected from a spray, pressurized-spray, atomized spray, wash, rinse, hose, shower, mist, aerosol, nebulizer, spray gun, jet-spray, vaporize, sprinkle hand-held spray, pressurized canister spray, containers with pump spray top, containers with pump device or mix, apply into commercial power spray units, and, integrated into water line pressure cleaning systems;
(iv) repeating the steps (i) to (iii), wherein the vertebrate is poultry, cattle, swine, ovine, equine animals, pet, domesticated animal, zoo animal, laboratory animal, a wild animal, or farm animal.

A method of non-horticulture food supply bio-security systems, wherein disinfecting and preventing the passage of microbial from a food vertebrate and food vertebrate product to first human (who handled the animal and/or animal parts during processing) and end human consumer comprising:

(i) a disinfectant or NCDAI comprising of:

at least two or more native substances; wherein the native substances may be selected from black cumin oil, carob powder, cardamom powder, fenugreek powder, moringa, Agaricus mushroom powder, micro-algae, insect protein powder, coriander powder, ginger powder, cinnamon powder, cranberry seed oil, and oregano oil; These can be used in powder, oil, extract form or any combination thereof;

(ii) applying the said composition during housing and slaughtering of the food-vertebrate and food vertebrate product animal onto innate surfaces, non-innate surfaces, waters, water-lines, air, vents, hatcheries, nest, physical support structures, incubators, premise, caged layer housing, broiler house, backyard poultry coups, HVAC, floors, bins, water line pressure and non-pressure cleaning system, ancillary structural fixtures and tools;

(iii) applying the said composition by one or more of the devices selected from a spray, sprinkle, shower, mist, aerosol, nebulizer, spray gun, jet-spray, vaporize, pressurized-spray, atomized spray, wash, rinse, hose, hand-held spray, pressurized canister spray, containers with pump spray top, containers with pump device or mix, apply into commercial power spray units, and, integrated into water-line pressure cleaning system;

repeating the steps (i) to (iii), wherein the vertebrate is poultry, cattle, swine, ovine, equine animals, farm animals, domesticated animals, sports animals, zoo animals, pets, laboratory animals, wild animals, and any combination of thereof.

A method of non-horticulture food supply bio-security systems, wherein disinfecting and preventing the passage of microbial zoonoses (may be between food-animals; between food-animals and non-food animals). From the first food-vertebrate and food-vertebrate product to second food vertebrate and food-vertebrate product to end human consumer comprising:

(i) a disinfectant or NCDAI comprising of:

at least two or more native substances; wherein the native substances may be selected from black cumin oil, carob powder, cardamom powder, fenugreek powder, moringa, Agaricus mushroom powder, micro-algae, insect protein powder, coriander powder, ginger powder, cinnamon powder, cranberry seed oil, and oregano oil; These may be used in powder, oil, extract, aerosol form and/or any combination thereof;

(ii) applying the said composition during housing and slaughtering of the food vertebrate and food vertebrate product animal onto innate surfaces, non-innate surfaces, waters, water-lines, air, vents, hatcheries, nest, physical support structures, incubators, premise, caged layer housing, broiler house, backyard poultry coups, HVAC, floors, bins, water line pressure and non-pressure cleaning system, ancillary structural fixtures, tools, etc., and any combination thereof;

(iii) applying the said composition by one or more of the devices selected from a spray, sprinkle, shower, mist, aerosol, nebulizer, spray gun, jet-spray, vaporize, pressurized-spray, atomized spray, wash, rinse, hose, hand-held spray, pressurized canister spray, containers with pump spray top, containers with pump device or mix, apply into commercial power spray units, and, integrated into water-line pressure cleaning system;

repeating the steps (i) to (iii), wherein the vertebrate is a poultry, cattle, swine, ovine, equine, farm animals, domesticated animals, sports animals, zoo animals, pets, laboratory animals, wild animals, and any combination of thereof.

Example: The presently disclosed subject matter is better understood from reading the following example and associated experimental data and results, along with the description of non-limiting embodiments mentioned herein. The example illustrated herein uses composition A and composition B, and a methodology for each experiment are described in each figure. The composition had a final total of volume of 40 gallons.

### Composition A

| Ingredient | Qty/lbs | Range (% wt/vol) |
|---|---|---|
| Coconut Oil | 61 | 18.3%-25.9% |
| Chia Flour | 50.3 | 12.3%-19.9% |
| Krill Oil | 50.2 | 17.4%-30.2% |
| Oregano Oil | 50.2 | 13.3%-19.8% |
| Fumed Silica | 24 | 2.4%-8.9% |
| Coriander Powder | 10.1 | 1.5%-7.8% |
| Cranberry Seed Oil | 8.5 | 1.2%-7.4% |
| Ginger Powder | 2.1 | 0.3%-1.8% |
| Black Cumin Oil | 1.9 | 0.3%-1.7% |
| Carob Powder | 2 | 0.2%-1.6% |
| Fenugreek Powder | 1.3 | 0.1%-1.2% |
| Cardamom Powder | 1.1 | 0.1%-1.1% |
| Agaricus Mushroom | 0.9 | 0.1%-1.0% |
| Powder | | |

### Composition B

| Ingredient | Range (% wt/wt) |
|---|---|
| Oregano Oil | 11.0 |
| Organic Certified Coconut oil | 24.5 |
| Elantria DHA 400 Algae | 18.8 |
| MCT Oil | 25.0 |
| Cranberry Seed oil | 3.5 |
| Coriander oil | 3. |
| Ginger oil | 1.0% |
| Black Cumin Oil | 1.0 |
| Carob oil | 1.0 |
| Fenugreek oil | 0.4 |
| Agaricus oil | 0.5% |
| Cinnamon oil | 0.8 |
| Lemon Oil | 9.5 |

### Composition C

| Ingredient | Range (% wt/wt) |
|---|---|
| Oregano Oil | 23.0% |
| Organic Certified Coconut oil | 20.8% |
| Elantria DHA 400 Algae | 15.0% |
| MCT Oil | 21.0% |
| Cranberry Seed oil | 3.7% |
| Coriander oil | 2.6% |
| Ginger oil | 1.4 |
| Black Cumin Oil | 0.5% |
| Carob oil | 0.5% |
| Fenugreek oil | 0.5% |
| Agaricus oil | 0.6% |
| Cinnamon oil | 1.0% |
| Lemon Oil | 10.4% |

All three compositions showed similar results; however, all data not shown compactness.

Microbials were tested in two independent laboratories, from the UMC-VMSL in Columbia, Mo., and microbe InoTech of St Louis. The microbes were provided by the two laboratories above and known potentially lethal antibiotic-resistant Salmonella species were provided by the CDC/DDID, Atlanta. Clostridium perfringens MRSA (methicillin resistant Staphylococcus aureus), Salmonella species. (S. typhimurim, S. heidelberg, S. senftenberg, S. hadar) (repeated with AMR (anti-microbial resistant) species of Salmonella to include S. typhimurim, S. heidelberg, these last 3 are from CDC and are different variants of the prior Salmonella. Chemicals were purchased from Fisher-Scientific, and herbs were purchased from numerous commercial sources such as American Botanicals.

The results presented herein for the compositions A, B, and C are unexpected, unpredictable, and of significant benefit for all vertebrates. Several compositions were tested, but only a few (including the three presented herein) showed compositions that provided the surprisingly unexpected results given herein. Quite unexpectedly, we observed that the composition inhibited all bacterial species, yeast, and fungi from surviving at 99.9-100% incident with an average of 10⁶-10⁸ log change, heralded by numerous industry professionals as ‘quite impressive’ and ‘exciting.’ The composition of this invention with the surprising and unpredictable anti-microbial activity contains non-toxic, side-effect free, all natural, essential plant oils, have never before been commercially available in the measured formulation of this invention. The compilation of the ingredients of the composition of this invention is useful in both preventative health maintenance and for active clinical illness in any vertebrate species, including humans. Thus, this invention is unique and novel.

An embodiment of the composition has anti-microbial activity against drug resistance microbes such as, but not limited to, AMR Salmonella species described herein and others.

Prepared microbial overnight cultures and adjusted the culture Optical Density (OD) to 1 (approximate 10⁸ CFU/ml), inoculated 10 μl (microliters) of the OD₆₀₀ of 1 culture dilutions into 10 ml of LB (approximately 10⁵ CFU/ml). Either Composition A, B, or C were added to each of the experimental cultures (100× final dilution) but not to the control experiments. The inoculated cultures were incubated in a shaking incubator at 37° C. and 200 rpm for 3 hours. The cultures were serially diluted and plated on L-Agar and then incubated at 37° C. overnight. On the next day, the numbers of colonies on each plate were counted.

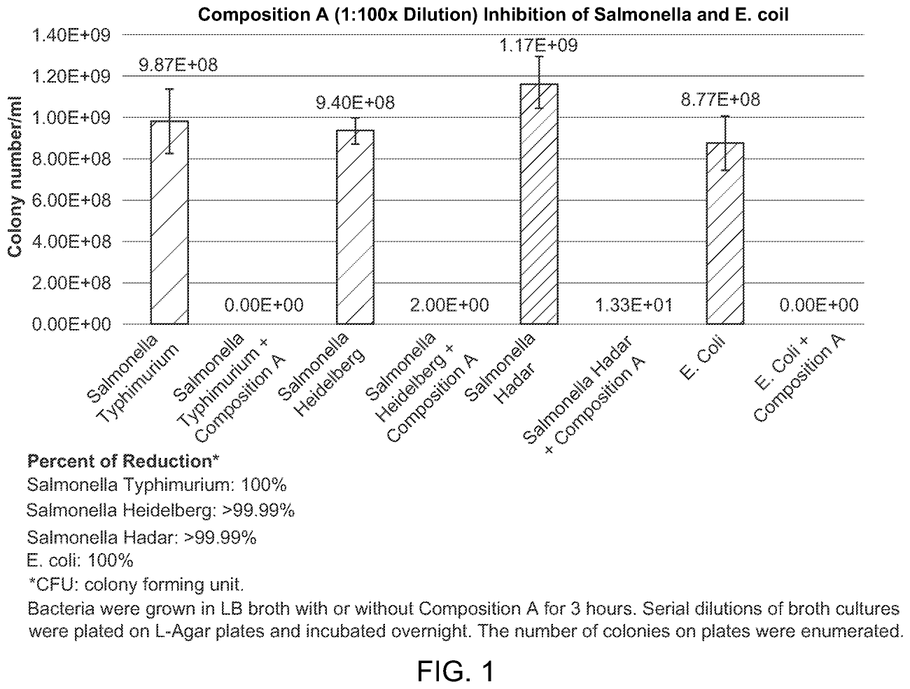

FIG. 1: Composition A/B/C (1:100× dilution) inhibition of Salmonella and E. coli: About 100% reduction for both Salmonella species and E. Coli, as shown in FIG. 1. An average of at least three different experiments.

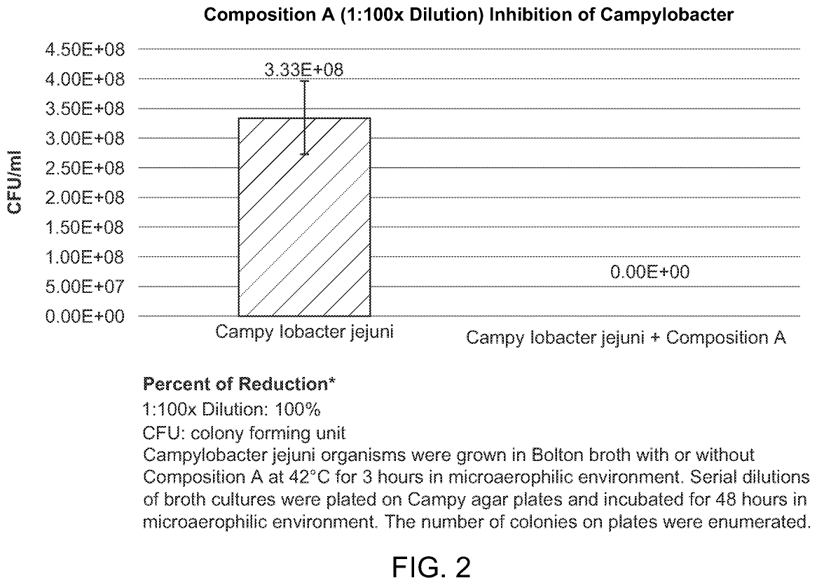

FIG. 2: Composition A/B/C (1:100× Dilution) Inhibition of Campylobacter jejuni. 100% reduction of Campylobacter jejuni, as shown in FIG. 2. An average of at least three different experiments

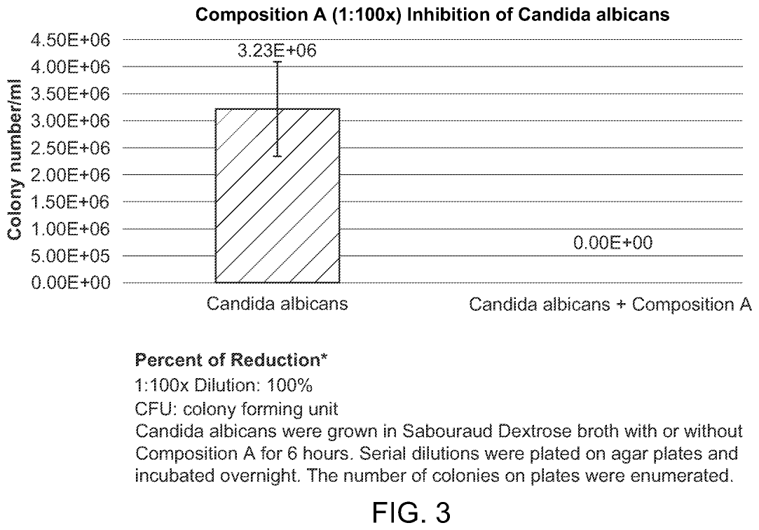

FIG. 3. Composition A/B/C (1:100× Dilution) Inhibition of Candida albicans. 100% reduction of Candida albicans, as shown in FIG. 3. An average of at least three different experiments

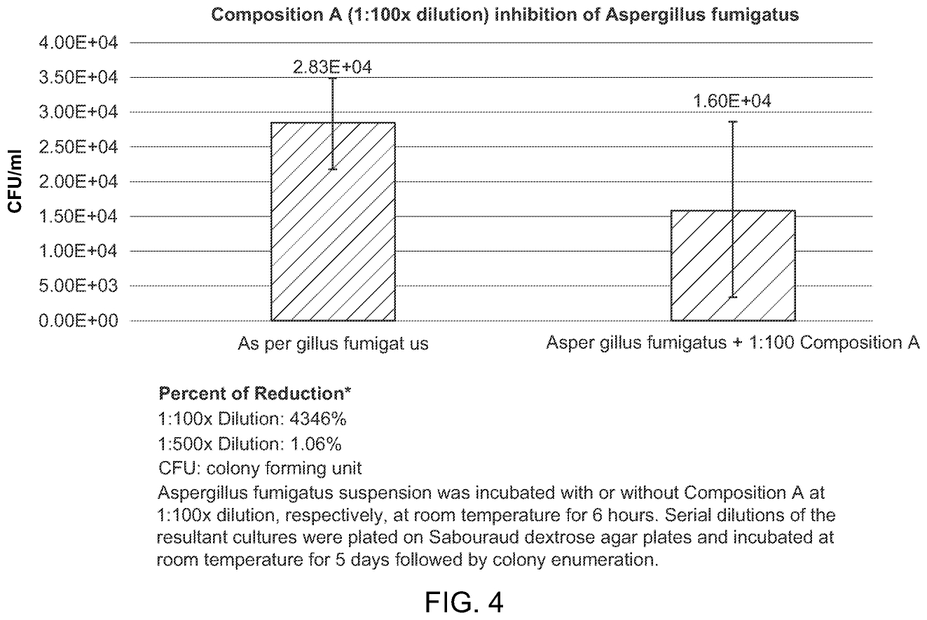

FIG. 4. Composition A/B/C Inhibition of Aspergillus fumigatus. Composition A/B at a dilution of 100× reduced the Aspergillus fumigatus by about 44%, an average of at least three different experiments, as shown in FIG. 4.

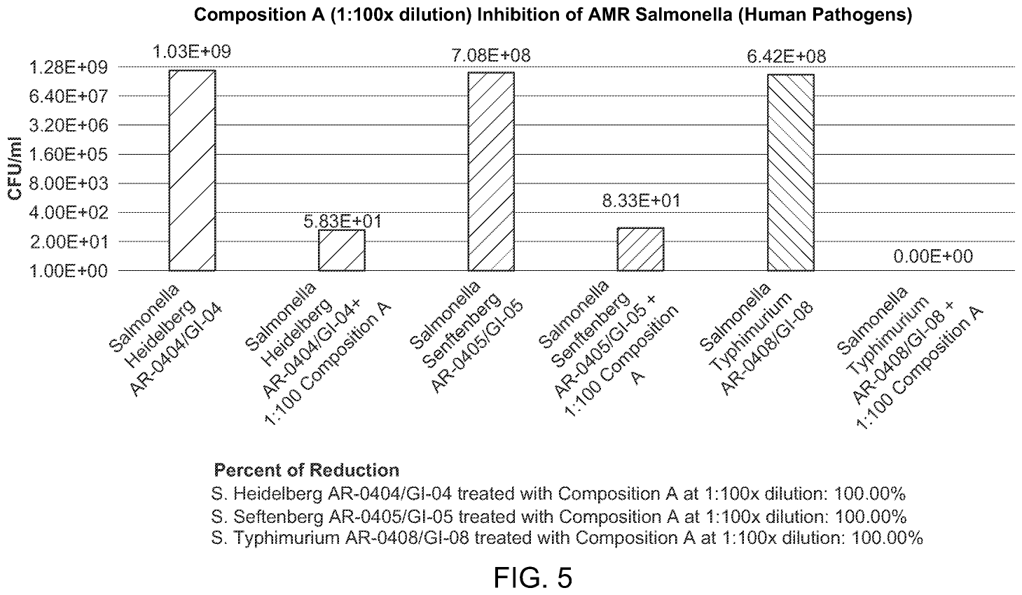

FIG. 5. Composition A/B/C Inhibition of (Anti-Microbial Resistance) AMR Salmonella. Even Salmonella species resistant to known anti-microbial exhibited significant inhibition of about 100%, an average of at least three different experiments. These human infecting AMR Salmonella cause serious diseases in humans and are highly resistant to several antibiotics. The results presented in this figure are unexpected and extremely beneficial in the treatment of drug-resistant bacterial infections in humans.

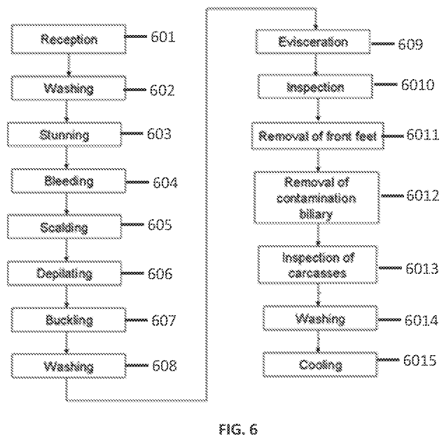

FIG. 6. Generalized Steps in Animal Slaughtering. The slaughtering program may be divided into the following subroutines, such as but not limited to, Clearance for cleaning, Assembling for cleaning, Removal of solid waste, Pre-rinsing with water, Application of detergent, Rinsing, Disinfection, Post rinsing, Post-treatment, and Disassembling after cleaning, etc., and any combination thereof. Shown herein are steps during animal slaughtering, starting with bringing the animals, i.e., reception 601, and getting ready for slaughtering 601-609. Steps 601 to 608 may be conducted in a non-clean or dirty environment. However, evisceration must be done in a clean and sanitary environment to prevent the transmission of new and/or old microbes to end human users. The new microbes may grow on the slaughtered animal or animal parts; whereas, old microbes may be microbes already present on outer surfaces or within the slaughtered animals. Thus, processes or steps 6010 to 6015 must be performed in a clean and aseptic condition. This invention's composition may be used in steps 601-6015, more specifically 602, 609, 6010, 6011, 6012, 6014, 6015, and/or any combination thereof. The composition may also be used during washing, disinfection, and cleaning of the facilities, equipment, machinery, instruments, human workers, clothing, protective equipment, animal housing, shipping animal and animal products, etc., and any combination thereof. Detergents may or may not be used during cleaning, disinfection, and/or washing of these steps, and the facilities, equipment, machinery, instruments, human workers, clothing, protective equipment, animal housing, etc., and any combination thereof. The detergents may be chemically synthesized and/or native and non-chemically, synthesized detergents such as anionic, cationic, non-ionic, etc., and any combination thereof.

It is to be understood that the above description is intended to be illustrative and not restrictive. For example, the above-described embodiments (and/or aspects thereof) may be used in combination with each other. In addition, many modifications may be made to adapt a particular situation, method, system device or material to the teachings of the various embodiments of the invention without departing from their scope. While the particulars and details described herein are intended to define the parameters of the various embodiments of the invention, the embodiments are by no means limiting and are exemplary embodiments. Many other embodiments will be apparent to those of skill in the art upon reviewing the above description. The scope of the various embodiments of the invention should, therefore, be determined with reference to the appended claims, along with the full scope of equivalents to which such claims are entitled.

This written description uses examples to disclose the various embodiments of the invention, including the best mode, and also to enable any person skilled in the art to practice the various embodiments of the invention, including making and using any devices or systems and performing any incorporated methods. The patentable scope of the various embodiments of the invention is defined by the claims, and may include other examples that occur to those skilled in the art. Such other examples are intended to be within the scope of the claims if the examples have structural elements or steps that do not differ from the literal language of the claims, or if the examples include equivalent structural elements or steps with insubstantial differences from the literal language of the claims.

What is claimed is:

1. A non-horticulture food supply bio-security system and compositions comprise of two or more native substances selected from a group consisting of chia seeds, carob, cardamom, fenugreek, moringa, Agaricus mushroom, micro-algae, insect protein, coconut/MCT oil, Elantria DHA 400 algae, cranberry seed, black cumin oil, oregano, carob, Agaricus mushroom, krill, coriander, ginger, cinnamon, micro-algae, lemon, cranberry seed oil, and oregano oil; wherein the composition is NCDAI.

2. The composition of claim 1, wherein the composition disinfects immediately on contact.

3. The composition of claim 1, wherein the composition has extended surface contact functional time of up to 0-28 days, 0-15, 0-16, 0-17, 0-18, 0-19, 0-20, 0-21, 0-22, 0-23, 0-24, 0-25, 0-26, 0-27, 0-28 days.

4. The composition of claim 1, wherein the composition has extended surface contact functional time greater than 28 days up to two years.

5. The composition of claim 1, wherein the composition disinfects inanimate surfaces, animate surfaces, waters, water-lines, air, vents, hatcheries, nest, physical support structures, incubators, premise, caged layer housing, broiler house, backyard poultry coups, HVAC, animal housing, hose, floors, bins, ancillary structural fixtures and tool, personnel skin, hair, nails, clothing, protective outerwear, inhalable nasal passages, oral cavity, slaughtering facilities, slaughtering equipment, slaughtering machinery, slaughtering instruments, slaughtering, packing, protective equipment shipping human workers, animal housing, shipping animal and animal products.

6. The composition of claim 1, wherein the composition is non-toxic, non-corrosive, non-irritant, inhalable, and ingestible.

7. The composition of claim 1, wherein the composition has a neutral environmental impact.

8. The composition of claim 1, wherein the composition has a positive environmental impact

9. The composition of claim 1, wherein the composition is non-foaming with cleaning properties.

10. The composition of claim 1, wherein the composition is packaged in one or more of polypropylene 8 oz hand sprayers, 1.6 kg jugs with a commercial hand pump sprayer, in 55-gallon drums with a universal adapter for large volume commercial sprayer units.

11. The composition of claim 1, wherein the composition has at least one or more selected from the group consisting of anti-coccidial, anti-mite, anti-arthropod, anti-insect, anti-fleas, anti-lice, anti-beetles, anti-bacterial, anti-yeast, anti-fungus, anti-viral, parasiticides, arthrocides, anti-microorganisms, anti-rickettsial, and pesticide.

12. The composition of claim 1, wherein the composition is non-oxidizing, non-reducing, non-flammable, non-explosive, non-reactive under ambient temperature, pressure, and environmental conditions; wherein the composition is non-brominating, without-aldehydes, and without-ammonia.

13. The composition of claim 1, wherein the composition is applied as one or more selected from a group consisting of a spray, pressurized-spray, atomized spray, wash, rinse, hand-held spray, hose, shower, mist, aerosol, nebulizer, spray gun, jet-spray, vaporize, sprinkle, commercial over-head sprinkler, ground sprinkler, pressurized canister spray, containers with pump spray top, containers with a pump device, commercial power spray units, and integrated into water line pressure cleaning system.

14. The composition of claim 1, wherein the composition is diluted with a solvent at the site of use.

15. The composition of claim 1, wherein the composition is at least a suspension, solution, or emulsion.

16. The composition of the claim 1, wherein the composition can be used in a vertebrate animal housing, topically on livestock, an egg cleanser, onto husbandry fixtures, cages, nest boxes, incubators, coops, flooring, walls, premise-penned outside yards, large commercial holding for broilers or layers, tool surfaces, slaughtering facilities, slaughtering equipment, slaughtering machinery, slaughtering instruments, slaughtering, packing, protective equipment shipping human workers, animal housing, shipping animal and animal products, and any combination of thereof.

17. A method of non-horticulture food supply bio-security systems, wherein disinfecting, NCDAI, and preventing the passage of microbial from a food-vertebrate, vertebrate products to human worker in the food supply chain comprising:
i) a disinfectant comprising of:
two or more native substances selected from a group consisting of chia seeds, carob, cardamom, fenugreek, moringa, Agaricus mushroom, micro-algae, insect protein, coconut/MCT oil, Elantria DHA 400 algae, cranberry seed, black cumin oil, oregano, carob, Agaricus mushroom, krill, coriander, ginger, cinnamon, micro-algae, lemon, cranberry seed oil, and oregano oil;
ii) applying the said composition onto innate surfaces, non-innate surfaces, waters, waterlines, air, personnel, personnel clothing, personnel equipment, personnel protective wear, vents, personnel skin, personnel hair, personnel nails, personnel nasal passages, personnel oral passage, hatcheries, nest, physical support structures, incubators, premise, caged layer housing, broiler house, backyard poultry coups, HVAC, floors, bins, water line pressure, non-pressure cleaning system ancillary structural fixtures and tools, slaughtering facilities, slaughtering equipment, slaughtering machinery, slaughtering instruments, slaughtering, packing, protective equipment shipping human workers, animal housing, shipping animal and animal products;
iii) applying the said composition by one or more of the devices selected from a group consisting of a spray, pressurized-spray, atomized spray, wash, rinse, hand-held spray, hose, shower, mist, aerosol, nebulizer, spray gun, jet-spray, vaporize, sprinkle, commercial over-head sprinkler, ground sprinkler, pressurized canister spray, containers with pump spray top, containers with a pump device, commercial power spray units, and integrated into water line pressure cleaning system;
iv) repeating the steps (i) to (iii), wherein the vertebrate is poultry, cattle, swine, ovine, equine animals, pet, domesticated animal, zoo animal, laboratory animal, a wild animal, or farm animal.

19) A method of non-horticulture food supply bio-security systems, wherein disinfecting and preventing the passage of microbial from a food-vertebrate and food vertebrate product to first human and end human consumer comprising:
ii) a disinfectant comprising of:
at least two or more native substances; wherein two or more native substances selected from a group consisting of chia seeds, carob, cardamom, fenugreek, moringa, Agaricus mushroom, micro-algae, insect protein, coconut/MCT oil, Elantria DHA 400 algae, cranberry seed, black cumin oil, oregano, carob, Agaricus mushroom, krill, coriander, ginger, cinnamon, micro-algae, lemon, cranberry seed oil, and oregano oil, wherein the composition is NCDAI;
ii) applying the said composition during housing and slaughtering of the food vertebrate and food vertebrate product animal onto innate surfaces, non-innate surfaces, waters, water-lines, air, vents, hatcheries, nest, physical support structures, incubators, premise, caged layer housing, broiler house, backyard poultry coups, HVAC, floors, bins, water line pressure and non-pressure cleaning system, ancillary structural fixtures and tools, slaughtering facilities, slaughtering equipment, slaughtering machinery, slaughtering instruments, slaughtering, packing, protective equipment shipping human workers, animal housing, shipping animal and animal products;
iii) applying the said composition by one or more of the devices selected from a group consisting of a spray, pressurized-spray, atomized spray, wash, rinse, hand-held spray, hose, shower, mist, aerosol, nebulizer, spray gun, jet-spray, vaporize, sprinkle, commercial over-head sprinkler, ground sprinkler, pressurized canister spray, containers with pump spray top, containers with a pump device, commercial power spray units, and integrated into water line pressure cleaning system;
iv) repeating the steps (i) to (iii), wherein the vertebrate is poultry, cattle, swine, ovine, equine animals, farm animals, domesticated animals, sports animals, zoo animals, pets, laboratory animals, wild animals, and any combination of thereof.

20) A method of non-horticulture food supply bio-security systems, wherein disinfecting and preventing the passage of microbial zoonoses from first food vertebrate and food-vertebrate product to second food vertebrate and food vertebrate product to end human consumer comprising:
iii) a disinfectant comprising of:
at least two or more native substances; wherein two or more native substances selected from a group consisting of chia seeds, carob, cardamom, fenugreek, moringa, Agaricus mushroom, micro-algae, insect protein, coconut/MCT oil, Elantria DHA 400 algae, cranberry seed, black cumin oil, oregano, carob, Agaricus mushroom, krill, coriander, ginger, cinnamon, micro-algae, lemon, cranberry seed oil, and oregano oil, wherein the composition is NCDAI;
ii) applying the said composition during housing and slaughtering of the food vertebrate and food vertebrate product animal onto innate surfaces, non-innate surfaces, waters, waterlines, air, vents, hatcheries, nest, physical support structures, incubators, premise, caged layer housing, broiler house, backyard poultry coups, HVAC, floors, bins, water line pressure and non-pressure cleaning system, ancillary structural fixtures, tools, slaughtering facilities, slaughtering equipment, slaughtering machinery, slaughtering instruments, slaughtering, packing, protective equipment shipping human workers, animal housing, shipping, animal and animal products;
iii) applying the said composition by one or more of the devices selected from a group consisting of a spray, pressurized-spray, atomized spray, wash, rinse, hand-held spray, hose, shower, mist, aerosol, nebulizer, spray gun, jet-spray, vaporize, sprinkle, commercial over-head sprinkler, ground sprinkler, pressurized canister spray, containers with pump spray top, containers with a pump device, commercial power spray units, and integrated into water line pressure cleaning system;
iv) repeating the steps (i) to (iii), wherein the vertebrate is a poultry, cattle, swine, ovine, equine, farm animals, domesticated animals, sports animals, zoo animals, pets, laboratory animals, wild animals and any combination of thereof.
